(19) United States
(12) Patent Application Publication
(10) Pub. No.: US 2012/0128698 A1
(43) Pub. Date: May 24, 2012

(54) DIAGNOSIS AND TREATMENT OF AUTOIMMUNE DEMYELINATING DISEASES

(76) Inventor: Menno van Lookeren Campagne, San Francisco, CA (US)

(21) Appl. No.: 13/381,304

(22) PCT Filed: Jul. 2, 2010
(86) PCT No.: PCT/US10/40992

§ 371 Date: Jan. 30, 2012

Related U.S. Application Data
(60) Provisional application No. 61/223,511, filed on Jul. 7, 2009.

(51) Int. Cl. A61K 39/395; A61P 37/02; A61P 37/00; C12Q 1/68; C07K 16/28

(52) U.S. Cl. 424/172.1; 435/6.12; 530/387.1

(57) ABSTRACT

The present invention concerns the diagnosis and treatment of autoimmune demyelinating diseases, such as multiple sclerosis (MS), by means of a CLM-I agonist.

### FIELD OF THE INVENTION

The present invention concerns the diagnosis and treatment of autoimmune demyelinating diseases, such as multiple sclerosis (MS).

### BACKGROUND OF THE INVENTION

Myeloid cells are the primary effector cells in autoimmune demyelinating diseases (Barnett et al., Multiple Sclerosis 12, 121-132, 2006; Benveniste, Journal of Molecular Medicine 75, 165-173, 1997). The CNS-infiltrating myeloid population consists of resident microglia, macrophages, inflammatory dendritic cells, plasmacytoid dendritic cells and conventional dendritic cells. MHCII and CD86 expressing myeloid dendritic cells (DCs) have received special attention due to their ability to reactivate antigen-specific T-cells (Deshpande et al., J Immunol 178, 6695-6699, 2007) and their involvement in epitope spreading leading to relapsing disease (Miller et al., J Immunol 178, 6695-6699, 2007). Next to serving as antigen presenting cells, inflammatory DCs directly regulate the local extracellular milieu by secreting proinflammatory cytokines and reactive oxygen intermediates, resulting in progressive demyelination and axon loss. The precursor cells of these TNF- and iNOS producing dendritic cells, also named TipDCs (Serbina et al., Immunity 19, 59-70, 2003) are inflammatory monocytes present in the circulation and recruited to areas of CNS inflammation. Converting inflammatory to type II anti-inflammatory monocytes by glatiramer acetate, a drug approved for MS, resulted in reversion of EAE severity (Weber et al., Nature Medicine 13, 935-943, 2007), further stressing an important role of these myeloid cells in regulating disease severity.

Other negative regulators of CNS infiltrating myeloid cells have previously been identified. For example, TREM-2 expressed on both resident microglia and infiltrating myeloid cells plays an important role in resolution of CNS inflammation by phagocytosis of myelin debris (Piccio et al., European Journal of Immunology 37, 1290-1301, 2007; Takahashi et al., PLoS Medicine 4, e124, 2007; Takahashi et al., The Journal of Experimental Medicine 201, 647-6572005, 2005). Similarly, IFNAR on myeloid cells down-modulates inflammatory responses in the CNS (Prinz et al., Immunity 28, 675-686, 2008). However, neither receptor is specific for inflammatory bone marrow-derived monocytes homing to the CNS.

CLM-1 (MAIR-V, LMIR-3, DigR2) was identified in search for myeloid specific cell surface receptors important for negative regulation of myeloid function. CLM-1 is part of the CMRF family, a multigene cluster on human chtromosome 17 with the mouse orthologues located on chromosome 11. All family members contain an extracellular IgV domain. Two family members in this cluster (CLM-1 and CLM-8) contain an ITIM sequence in the intracellular domain, the remainder have charged residues in the transmembrane region that may serve to recruit signaling adapters. CLM-1, the murine orthologue of human CD300f (Clark et al., Trends in Immunology 30, 209-217, 2009), was first described as a negative regulator of osteoclastogenesis (Chung et al., J. Immunol 171, 6541-6548, 2003). Subsequent studies have shown that CLM-1 serves an inhibitory role in Fc-receptor-mediated cell responses (Alvarez-Errico et al., The Journal of Experimental Medicine 206, 595-606, 2004; Fujimoto et al., International Immunology 18, 1499-1508, 2006). A biological role in autoimmune disease so far has not been described.

### SUMMARY OF THE INVENTION

The present invention is based, at least in part, in the identification of CLM-1, as a negative regulator of inflammatory DCs activity in the CNS by suppressing release of inflammatory cytokines and reactive oxygen species. Thus, CLM-1 is identified herein as a myeloid specific negative regulator of CNS inflammation and demyelination.

In one aspect, the invention concerns a method for the treatment of a demyelinating disease in a mammalian subject comprising administering to said subject an effective amount of a CLM-1 agonist.

In another aspect, the invention concerns a pharmaceutical composition for the treatment of a demyelinating disease, comprising an effective amount of a CLM-1 agonist in admixture with a pharmaceutically acceptable excipient.

In yet another aspect, the invention concerns the use of an effective amount of a CLM-1 agonist in the preparation of a medicament for the treatment of a demyelinating disease.

In a further aspect, the invention concerns a CLM-1 agonist for the treatment of a demyelinating disease.

In a still further aspect, the invention concerns a method for the diagnosis of a demyelinating disease comprising detecting a defect in the function of CLM-1.

In an additional aspect, the invention concerns a kit comprising a CLM-1 agonist and instructions for the treatment of a demyelinating disease.

In all aspect, the invention specifically includes the following embodiments:

In one embodiment, the mammalian subject is a human.

In another embodiment, the demyelinating disease is a demyelinating autoimmune disease.

In yet another embodiment, the demyelinating autoimmune disease affects the central nervous system (CNS).

In a further embodiment, the demyelinating autoimmune disease is selected from the group consisting of multiple sclerosis (MS), relapsing remitting MS (RRMS), primary and secondary progressing forms of MS, progressice relapsing forms of MS, encephalomyelitis, leukoencephalitis, transverse myelitis, neuromyelitis optica (Devic's disease), and optic neuritis.

In a still further embodiment, the demyelinating autoimmune disease is MS.

In a different embodiment, the demyelinating autoimmune disease affects the periopheral nervous system, including, without limitation, acute inflammatory demyelinating polyneuropathy (AIDP; Guillain-Barre syndrome); chronic inflammatory demyelinating polyneuropathy; anti-MAG peripheral neuropathy; and Motor and Sensory Neuropathy (HMSN) (also known as Hereditary Sensorimotor Neuropathy (HSMN), or Peroneal Muscular Atrophy, and Charcot-Marie-Tooth Disease).

In another embodiment, the CLM-1 agonist is an agonist anti-CLM-1 antibody.

### BRIEF DESCRIPTION OF THE DRAWINGS

FIG. 1. CLM-1 is expressed on inflammatory dendritic cells in CNS inflammatory lesions

(A) Increased CLM-mRNA transcripts in spinal chord at peak of disease. (B) Absence of CLM-1 expression on CNS resident CD11b+ cells. (C) CLM-1 expression on CD11bCD11c+ myeloid cells (D) CLM-1 CD11c co-expressing DCs in CNS inflammatory lesions at peak disease (thoracic, dorsal horn). (E) CLM-1 expressing DCs express iNOS and TNFα. Values are expressed as mean ±S.D. Scale bar in (D) is 50 μm.

FIG. 2. CLM-1 is expressed on inflammatory monocytes and dendritic cells

(A) CLM-1 is expressed on Cx3cr1^{lo} CD11c⁺ Ly6^{hi} positive inflammatory monocytes, but no on Cx3cr1^{hi} conventional DC precursors (B) CLM-1 is expressed on radiation-sensitive bone-marrow derived cells but not on irradiation-resistant CNS resident microglia. (C) CLM-1 expression of Cx3cr1^{lo} inflammatory DCs but not on Cx3cr1^{hi} microglia (D) Cx3cr1 and CLM-1 expression on a spinal chord section (thoracic) 14 days after immunization. Co-staining is observed in the periphery at meninges (arrowheads) whereas Cx3cr1hi macroglia (arrows) do not carry CLM-1. Scale bars: B (100 μm), D (50 μm)

FIG. 3. Lack of CLM-1 or treatment with a CLM-1 fusion protein leads to enhanced EAE

(A) Absence of CLM-1 protein expression in bone marrow-derived DCs obtained from CLM-1 knock out (ko) mice (left panel). Similar levels of MHC II and CD86 on DCs obtained from spinal chord at peak of disease (right panel). (B) Lack of CLM-1 staining, preserved morphology and similar inflammatory cell numbers in CLM-1 wt compared to ko mice. (C) Increased disease severity in CLM-1 ko mice or (D) CLM-1 wt mice treated with a CLM-1-Fc fusion protein. Scale bar in (B) is 50 μm.

FIG. 4. CLM-1 absence does not affect T-cell priming

(A) Proliferation and cytokine responses of re-stimulated antigen specific peripheral lymph node T-cells is similar in CLM-1 wt and ko mice. (B) T-cells from CLM-1 ko or wt donor mice induce similar disease in wt recipients (left panel). T cells from a CLM-1 wt donor induce increased disease severity in CLM-1 ko recipients compared to CLM-1 wt recipients (right panel).

FIG. 5. CLM-1 regulated release of myeloid—but not T-cell specific inflammatory mediators

(A) No difference in the number of Th1, Th17 and regulatory T-cells upon re-activation of MOG reactive spinal chord T-cells obtained from immunized CLM-1 wt and ko mice (B) Increased DC activation in CLM-1 wt and ko myeloid cells obtained from CNS inflammatory lesions. *p<0.01.

FIG. 6. CLM-1 regulates autoimmune demyelination

(A) Deconvolution image of CLM-1 positive cells and MOG positive myelin in a CNS lesion. (B) and (C): increased demyelination (indicated by the area marked with a white line in B and quantified in C) in CLM-1 ko compared to wt mice.

FIG. 7. Amino acid sequences of mouse (SEQ ID NO: 1) and human (SEQ ID NO: 2) CLM-1 polypeptides.

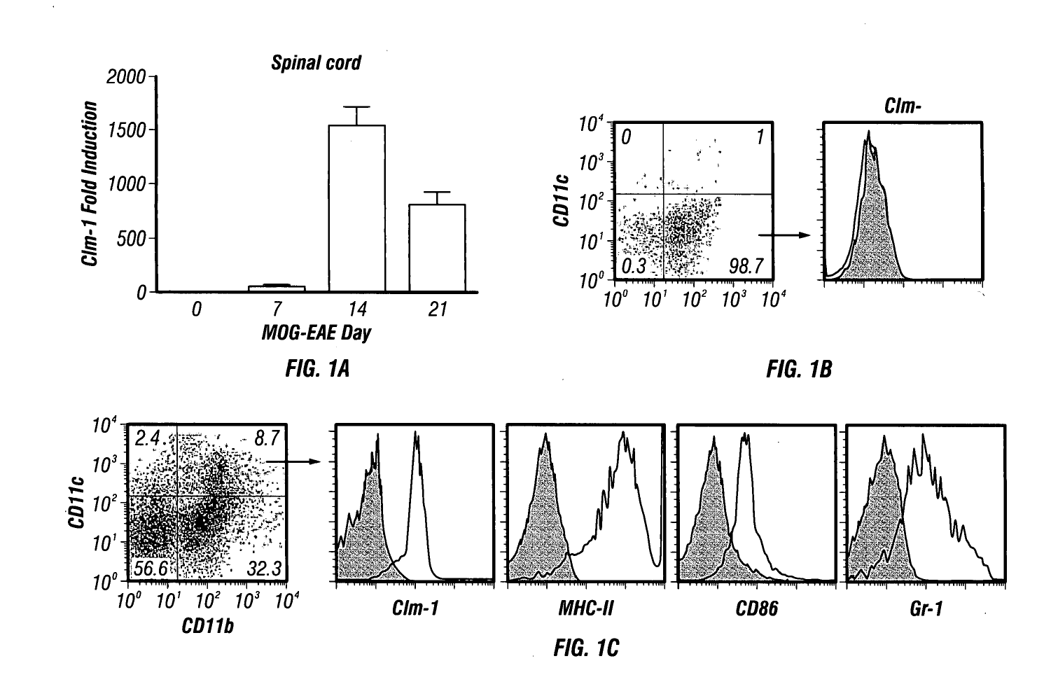

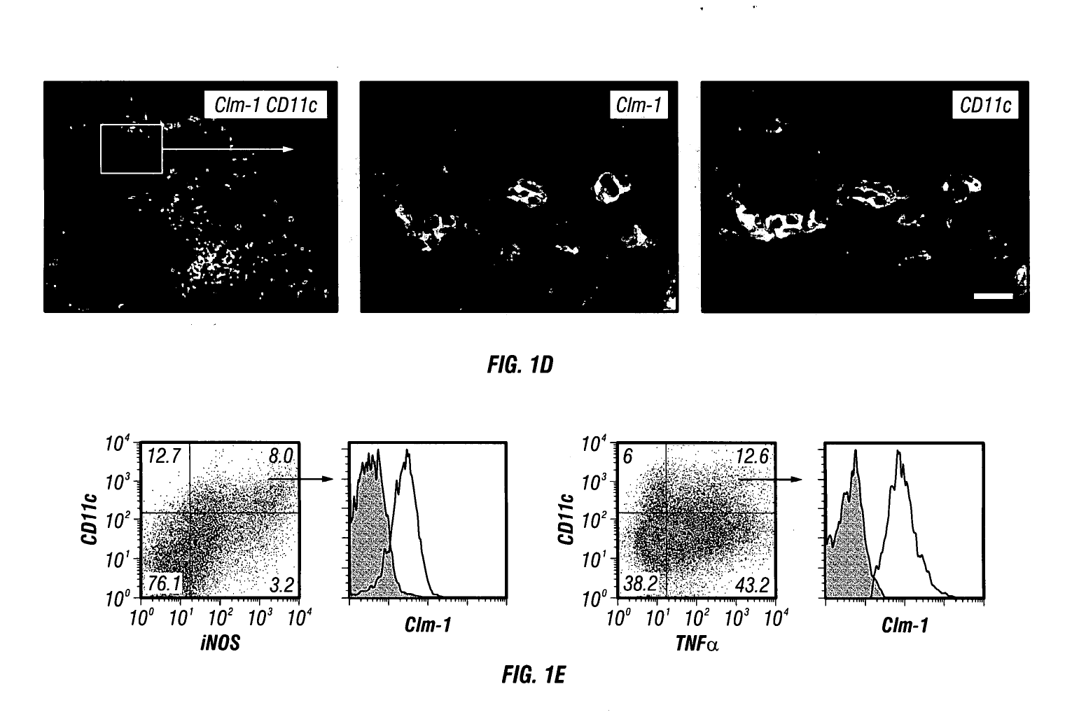

Supplemental FIG. 1. Strategy of targeted disruption of the mouse Clm-1 gene.

ES cells with replacement of Clm-1 exon-1 with the neomycin resistance gene were generated by homologous recombination. The structures of the targeted region of the Clm-1 gene are shown. E1 and E2 indicate exon 1 and exon 2 of the Clm-1 gene. The locations of the probes (5′ and 3′) used to screen the ES clones by Southern blotting are shown.

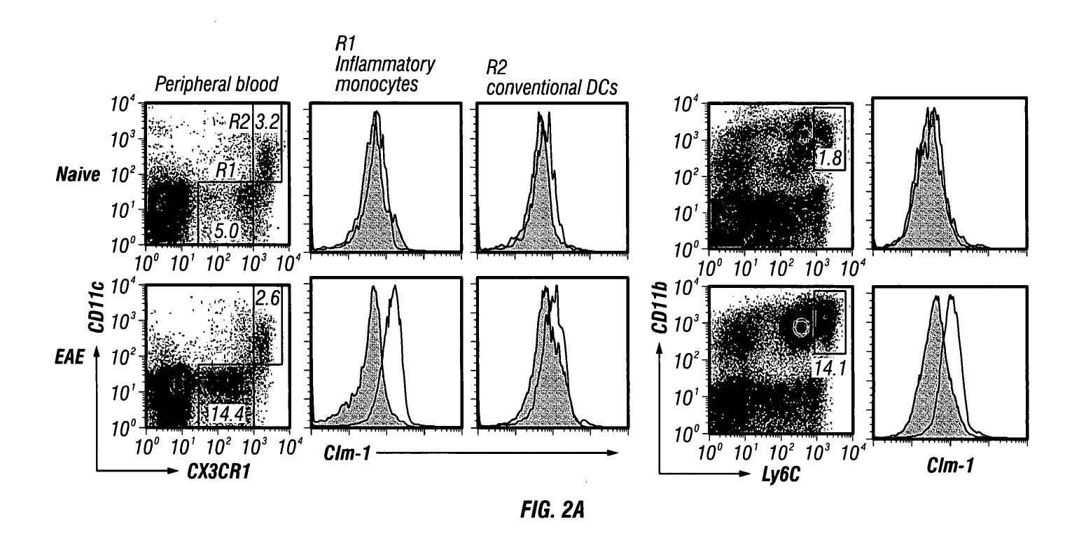

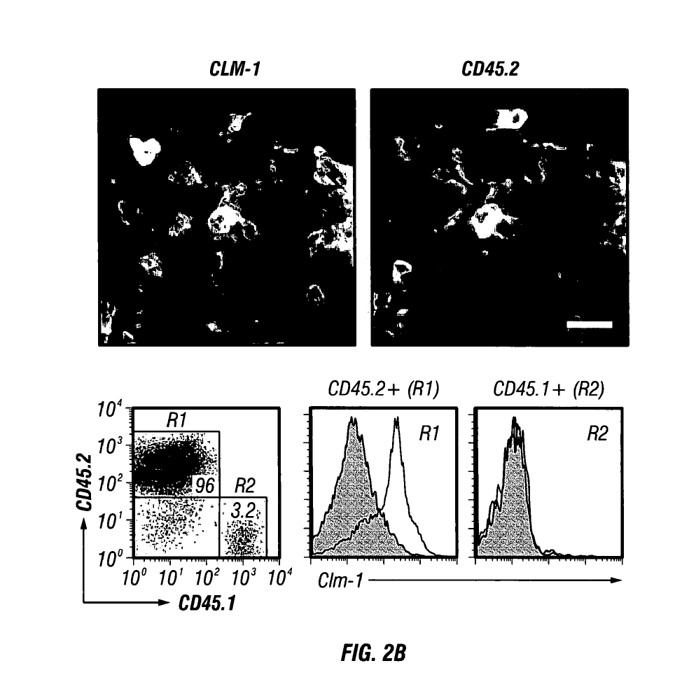

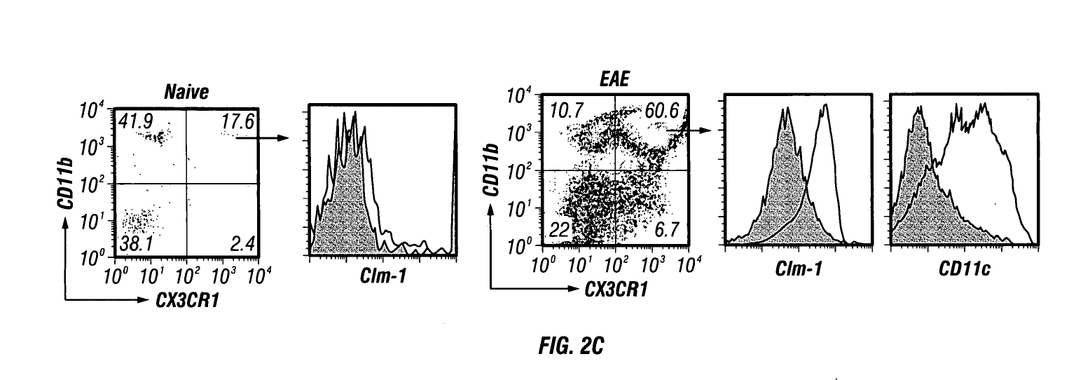

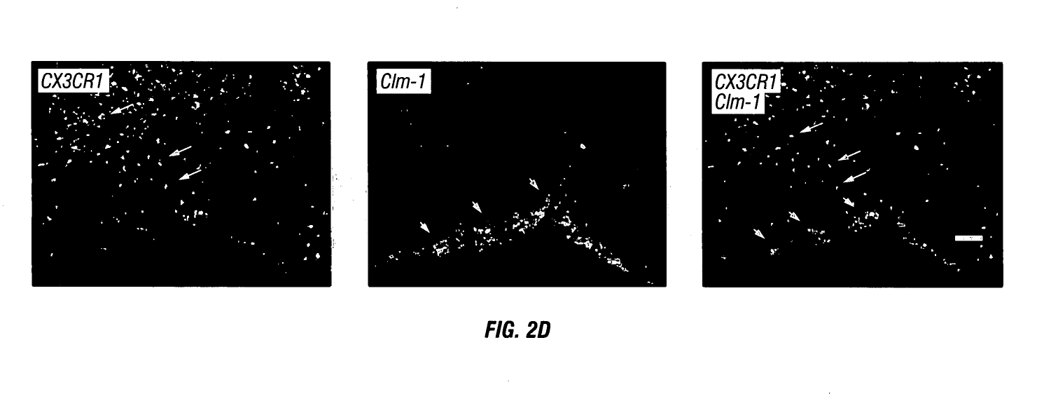

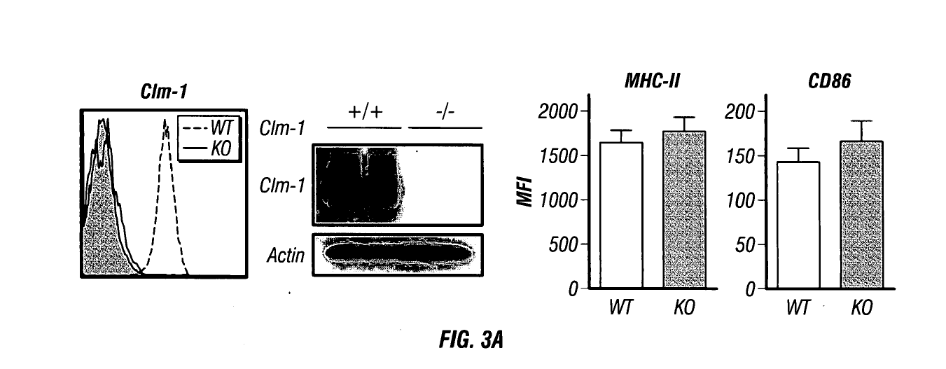

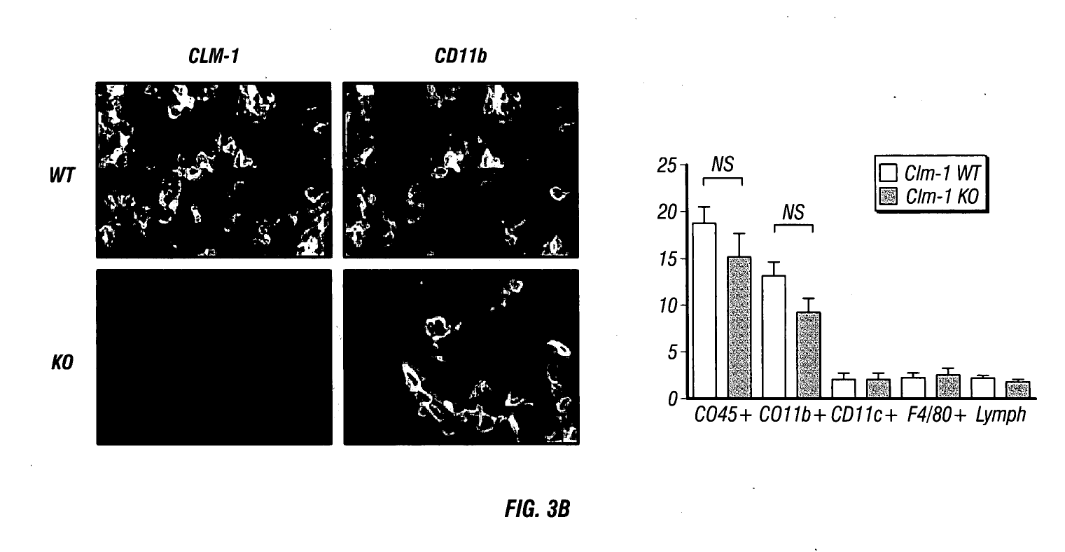

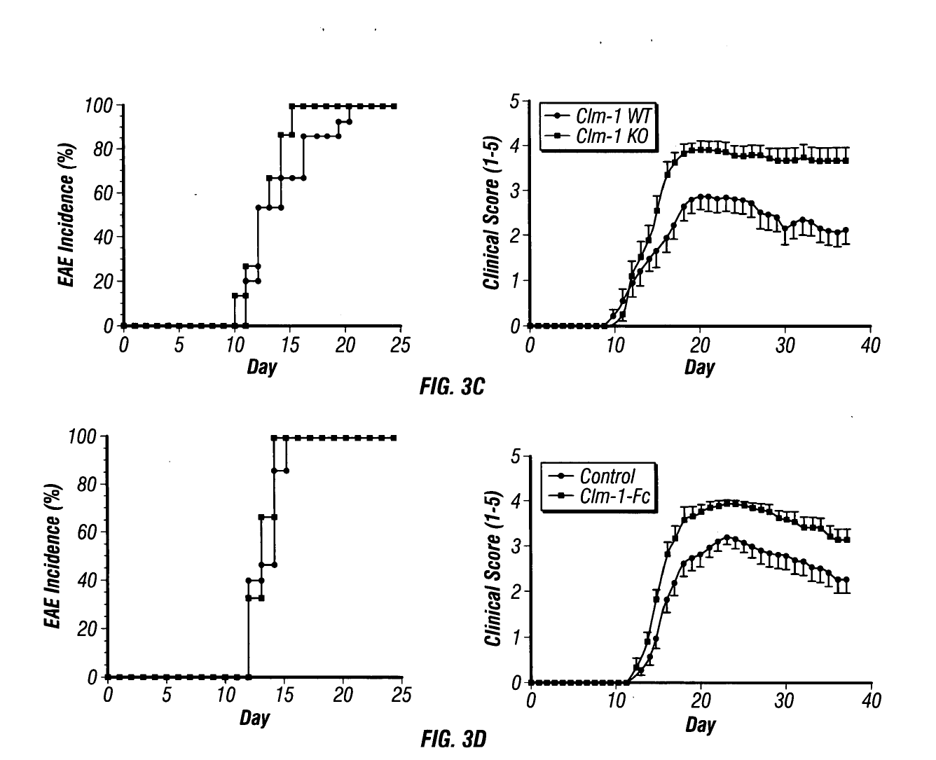

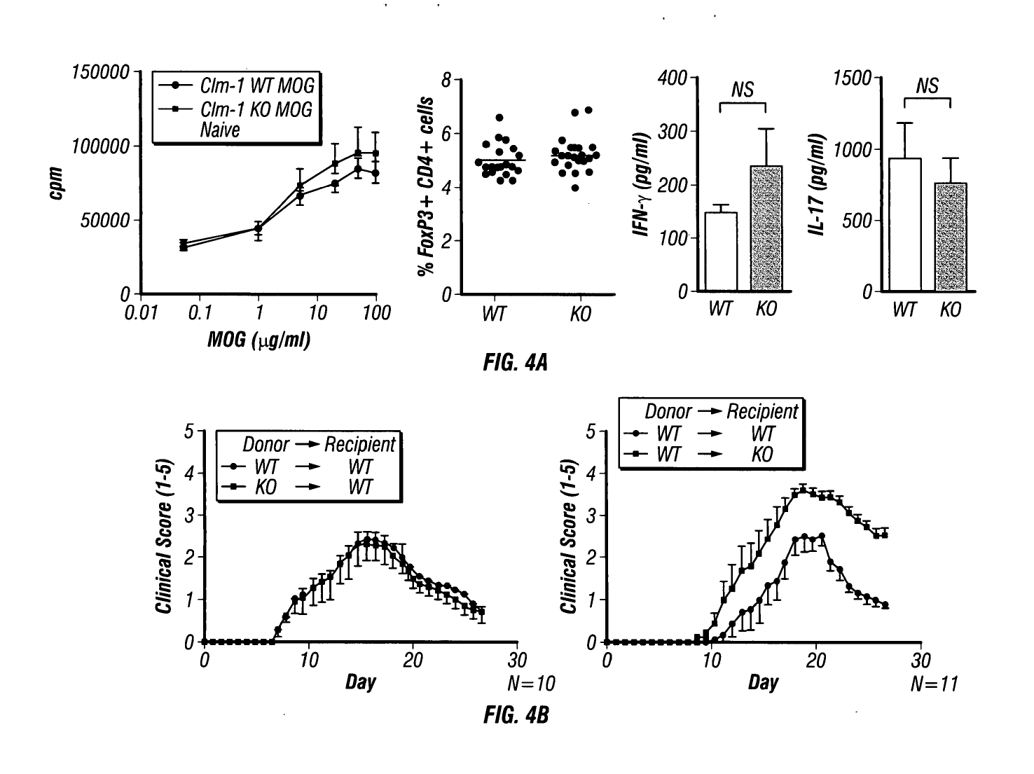

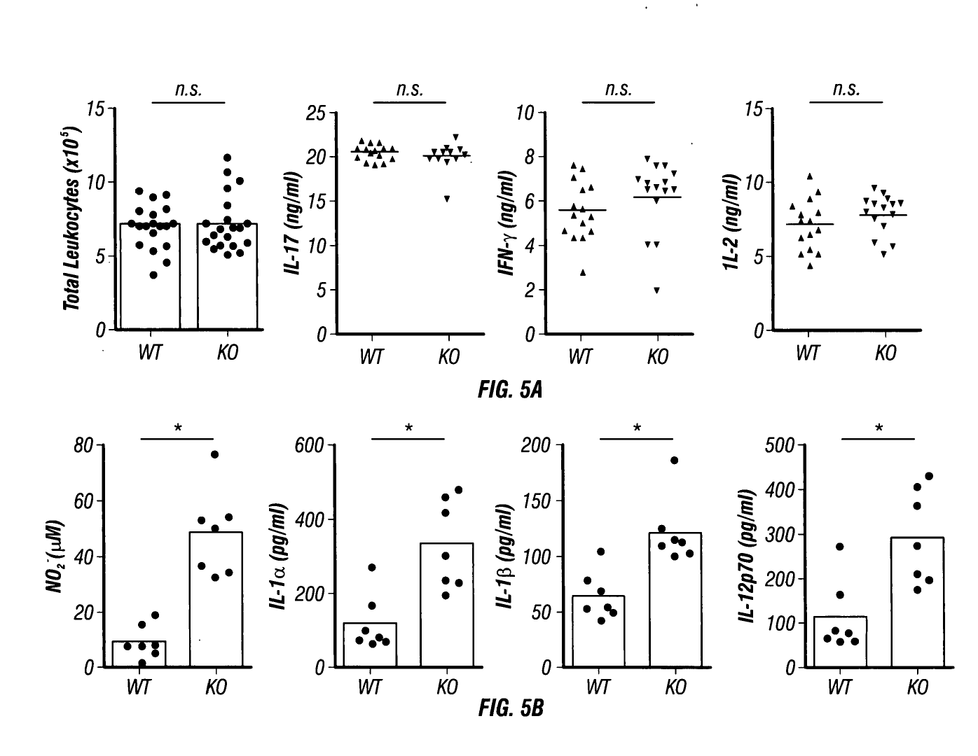

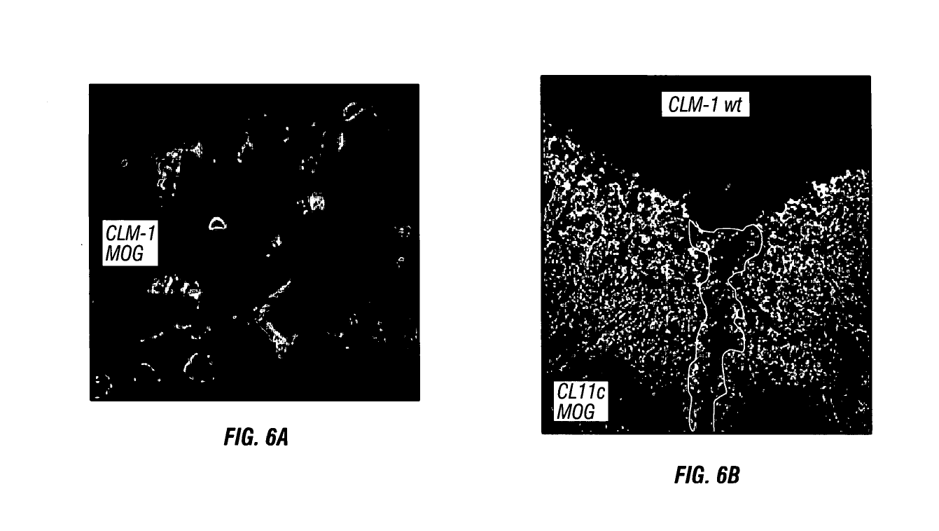

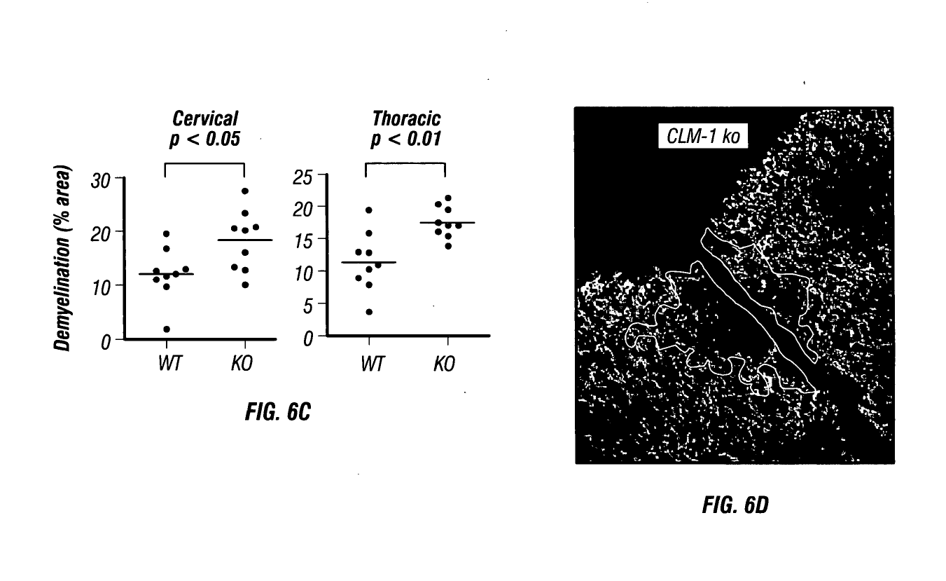

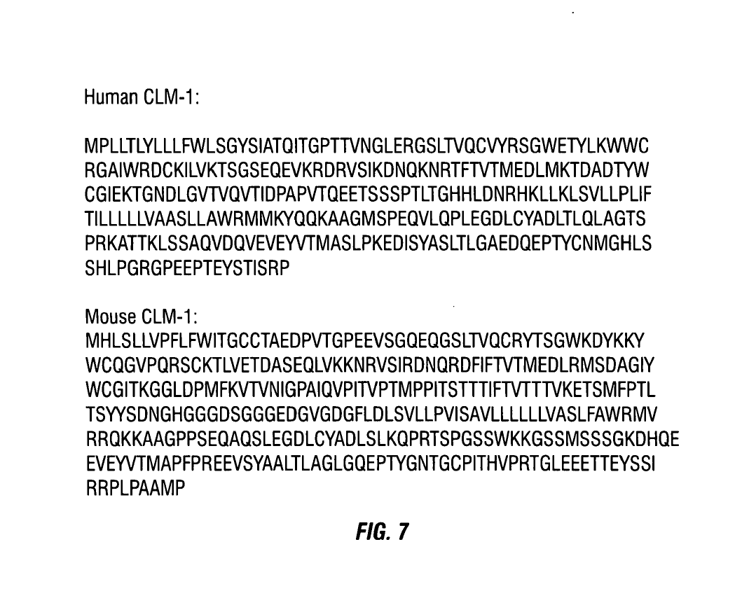

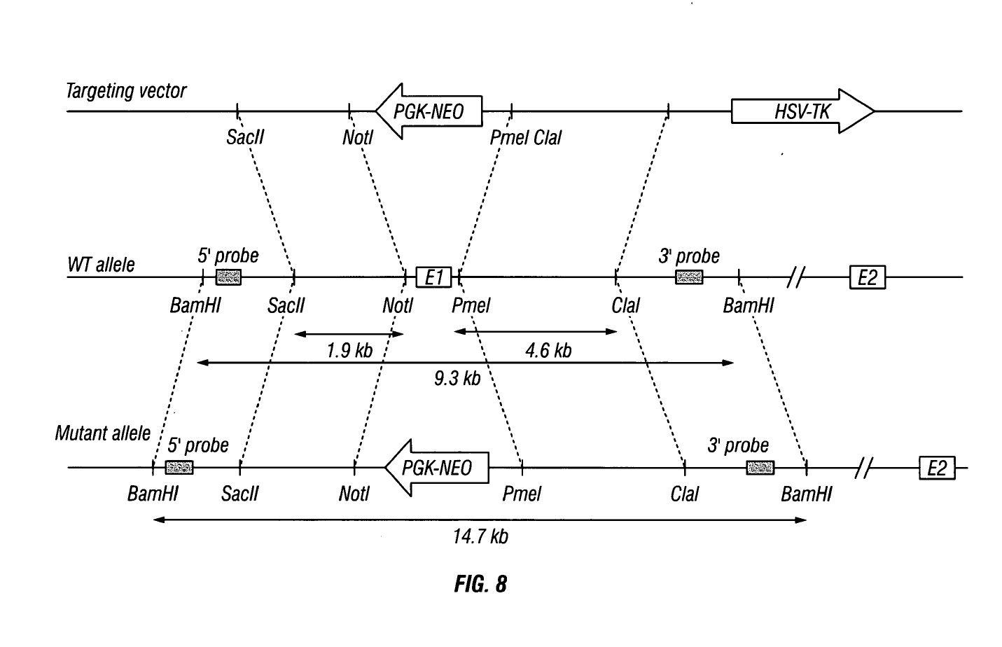

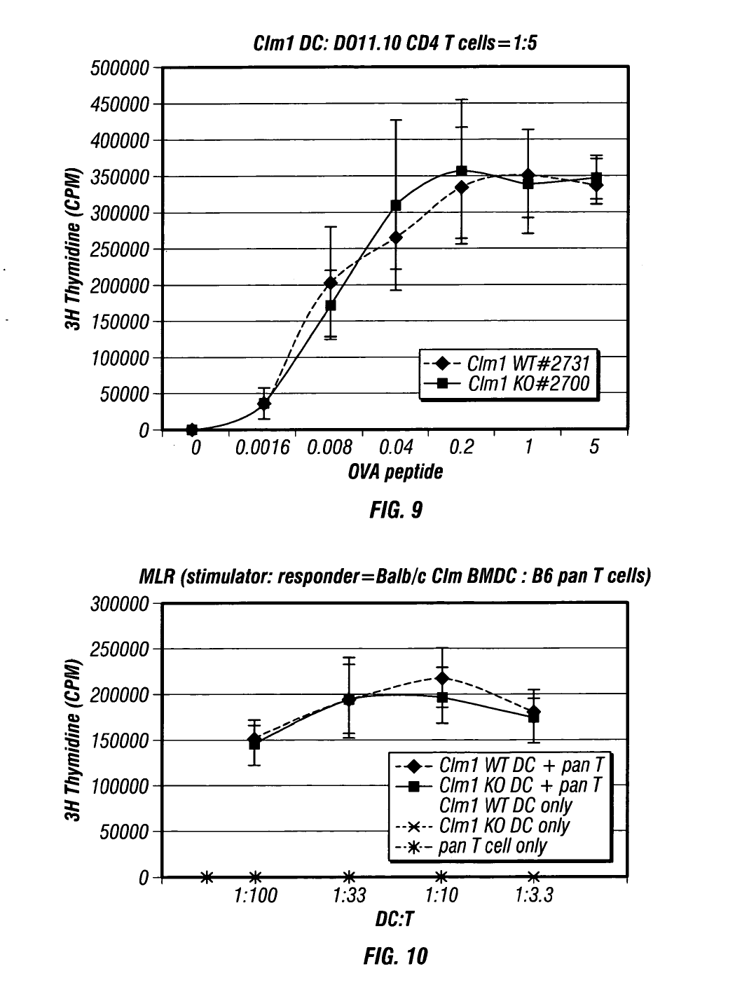

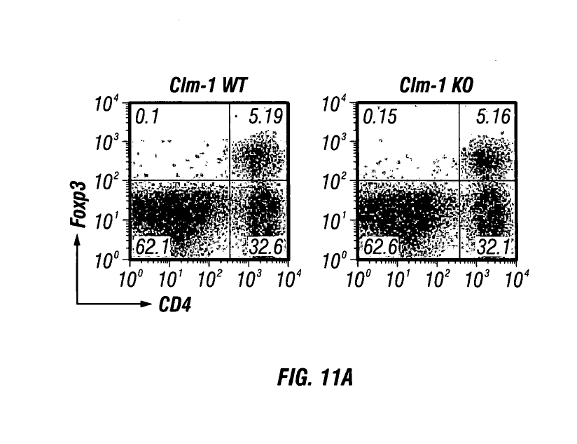

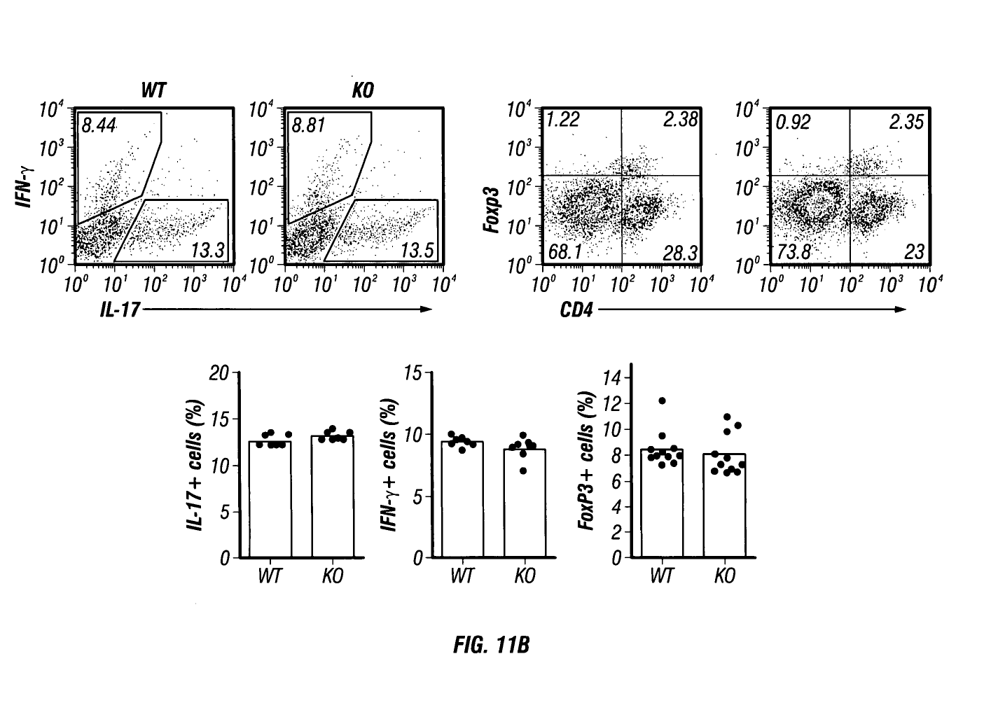

Supplemental FIG. 2. CLM-1 does not influence T-cell proliferation.

(A) T cells obtained from OVA transgenic T-cells were incubated with bone marrow-derived dendritic cells obtained from CLM-1 wt or ko mice in the presence of increasing concentrations of OVA peptide (B) Mixed Lymphocyte Reaction. Bone-marrow dendritic cells obtained from CLM-1 wt or ko mice on a Balb/c background were incubated with various ratios of T cells obtained from mice on a C57B1/6 background. Proliferation was reflected by the amount of H3 thymidine incorporation.

Supplemental FIG. 3. (A) Clm-1 does not influence regulatory T-lymphocyte generation in peripheral lymph nodes. (B) Clm-1 does not influence polarization of T-lymphocytes in the CNS.

### DETAILED DESCRIPTION OF THE PREFERRED EMBODIMENTS

### I. Definitions

The terms “CLM-1” and “Cmrf-Like Molecule-1” (also known as MAIR-V, LMIR-3, DigR2 and IgSF13) are used interchangeably herein to refer to a native sequence mammalian CLM-1 receptor, specifically including without limitation the mouse CLM-1 polypeptide of SEQ ID NO: 1 (NCBI CAM21607) and its human ortholog of SEQ ID NO: 2 (NCBI AAH28188, also known as CD300f, IREM1, IgSF13, 35-L5, and CMRF-35A5), as well as naturally occurring variants thereof. For further details and nomenclature see Clark et al., 2009, supra.

A “native sequence” polypeptide is one which has the same amino acid sequence as a polypeptide (e.g., ErbB receptor or ErbB ligand) derived from nature. Such native sequence polypeptides can be isolated from nature or can be produced by recombinant or synthetic means. Thus, a native sequence polypeptide can have the amino acid sequence of naturally occurring human polypeptide, murine polypeptide, or polypeptide from any other mammalian species.

The term “amino acid sequence variant” refers to polypeptides having amino acid sequences that differ to some extent from a native sequence polypeptide. Ordinarily, amino acid sequence variants will possess at least about 70% homology with at least one receptor binding domain of a native ErbB ligand or with at least one ligand binding domain of a native ErbB receptor, and preferably, they will be at least about 80%, more preferably at least about 90% homologous with such receptor or ligand binding domains. The amino acid sequence variants possess substitutions, deletions, and/or insertions at certain positions within the amino acid sequence of the native amino acid sequence.

“Homology” is defined as the percentage of residues in the amino acid sequence variant that are identical after aligning the sequences and introducing gaps, if necessary, to achieve the maximum percent homology. Methods and computer programs for the alignment are well known in the art. One such computer program is “Align 2”, authored by Genentech, Inc., which was filed with user documentation in the United States Copyright Office, Washington, D.C. 20559, on Dec. 10, 1991.

The term “antibody” herein is used in the broadest sense and specifically covers monoclonal antibodies, polyclonal antibodies, multispecific antibodies (e.g. bispecific antibodies) formed from at least two intact antibodies, and antibody fragments, so long as they exhibit the desired biological activity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc) and human constant region sequences.

“Antibody fragments” comprise a portion of an intact antibody, preferably comprising the antigen-binding or variable region thereof Examples of antibody fragments include Fab, Fab′, F(ab′)₂, and Fv fragments; diabodies; linear antibodies; single-chain antibody molecules; and multispecific antibodies formed from antibody fragment(s).

An “intact” antibody is one which comprises an antigen-binding variable region as well as a light chain constant domain (C_{L}) and heavy chain constant domains, C_{H}1, C_{H}2 and C_{H}3. The constant domains may be native sequence constant domains (e.g. human native sequence constant domains) or amino acid sequence variant thereof. Preferably, the intact antibody has one or more effector functions.

Antibody “effector functions” refer to those biological activities attributable to the Fc region (a native sequence Fc region or amino acid sequence variant Fc region) of an antibody. Examples of antibody effector functions include Clq binding; complement dependent cytotoxicity; Fc receptor binding; antibody-dependent cell-mediated cytotoxicity (ADCC); phagocytosis; down regulation of cell surface receptors (e.g. B cell receptor; BCR), etc.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, Fcγ RH and FcγRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. NoS. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H-} V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994), Anti-ErbB2 antibody scFv fragments are described in WO93/16185; U.S. Pat. No. 5,571,894; and U.S. Pat. No. 5,587,458.

The term “diabodies” refers to small antibody fragments with two antigen-binding sites, which fragments comprise a variable heavy domain (V_{H}) connected to a variable light domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An antibody “which binds” an antigen of interest is one capable of binding that antigen with sufficient affinity such that the antibody is useful as a therapeutic agent in targeting a cell expressing the antigen.

The term “demyelinating disease” is used herein to refer to any disease of the nervous system in which the myelin sheath of neurons is damaged. The definition includes both diseases that affect the integrity of the oligodendrocyte and its ability to produce and maintain myelin and diseases that directly damage the myelin sheath. Such diseases disturb conduction in myelinated white matter pathways and produce a broad array of motor, sensory, and cognitive dysfunctions, including impairment in sensation, movement, cognition, and/or other functions depending on which nerves are involved, including nerves of the central nervous system (CNS) and peripheral nerves.

An “autoimmune disease” herein is a disease or disorder arising from and directed against an individual's own tissues or a co-segregate or manifestation thereof or resulting condition therefrom. Examples of autoimmune diseases or disorders include, but are not limited to arthritis (rheumatoid arthritis, juvenile rheumatoid arthritis, osteoarthritis, psoriatic arthritis, and ankylosing spondylitis), psoriasis, dermatitis including atopic dermatitis; chronic idiopathic urticaria, including chronic autoimmune urticaria, polymyositis/dermatomyositis, toxic epidermal necrolysis, systemic scleroderma and sclerosis, responses associated with inflammatory bowel disease (IBD) (Crohn's disease, ulcerative colitis), and IBD with co-segregate of pyoderma gangrenosum, erythema nodosum, primary sclerosing cholangitis, and/or episcleritis), respiratory distress syndrome, including adult respiratory distress syndrome (ARDS), meningitis, IgE-mediated diseases such as anaphylaxis and allergic rhinitis, encephalitis such as Rasmussen's encephalitis, uveitis, colitis such as microscopic colitis and collagenous colitis, glomerulonephritis (GN) such as membranous GN, idiopathic membranous GN, membranous proliferative GN (MPGN), including Type I and Type II, and rapidly progressive GN, allergic conditions, eczema, asthma, conditions involving infiltration of T cells and chronic inflammatory responses, atherosclerosis, autoimmune myocarditis, leukocyte adhesion deficiency, systemic lupus erythematosus (SLE) such as cutaneous SLE, lupus (including nephritis, cerebritis, pediatric, non-renal, discoid, alopecia), juvenile onset diabetes, multiple sclerosis (MS) such as spino-optical MS, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including Large Vessel vasculitis (including Polymyalgia Rheumatica and Giant Cell (Takayasu's) Arteritis), Medium Vessel vasculitis (including Kawasaki's Disease and Polyarteritis Nodosa), CNS vasculitis, and ANCA-associated vasculitis , such as Churg-Strauss vasculitis or syndrome (CSS)), aplastic anemia, Coombs positive anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome, myasthenia gravis, antigen-antibody complex mediated diseases, anti-glomerular basement membrane disease, anti-phospholipid antibody syndrome, allergic neuritis, Bechet disease, Castleman's syndrome, Goodpasture's Syndrome, Lambert-Eaton Myasthenic Syndrome, Reynaud's syndrome, Sjorgen's syndrome, Stevens-Johnson syndrome, solid organ transplant rejection (including pretreatment for high panel reactive antibody titers, IgA deposit in tissues, and rejection arising from renal transplantation, liver transplantation, intestinal transplantation, cardiac transplantation, etc.), graft versus host disease (GVHD), pemphigoid bullous, pemphigus (including vulgaris, foliaceus, and pemphigus mucus-membrane pemphigoid), autoimmune polyendocrinopathies, Reiter's disease, stiff-man syndrome, immune complex nephritis, IgM polyneuropathies or IgM mediated neuropathy, idiopathic thrombocytopenic purpura (ITP), thrombotic throbocytopenic purpura (TTP), thrombocytopenia (as developed by myocardial infarction patients, for example), including autoimmune thrombocytopenia, autoimmune disease of the testis and ovary including autoimune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM), including pediatric IDDM, and Sheehan's syndrome; autoimmune hepatitis, Lymphoid interstitial pneumonitis (HIV), bronchiolitis obliterans (non-transplant) vs NSIP, Guillain-Barre Syndrome, Berger's Disease (IgA nephropathy), primary biliary cirrhosis, celiac sprue (gluten enteropathy), refractory sprue with co-segregate dermatitis herpetiformis, cryoglobulinemia, amylotrophic lateral sclerosis (ALS; Lou Gehrig's disease), coronary artery disease, autoimmune inner ear disease (AIED), autoimmune hearing loss, opsoclonus myoclonus syndrome (OMS), polychondritis such as refractory polychondritis, pulmonary alveolar proteinosis, amyloidosis, giant cell hepatitis, scleritis, monoclonal gammopathy of uncertain/unknown significance (MGUS), peripheral neuropathy, paraneoplastic syndrome, channelopathies such as epilepsy, migraine, arrhythmia, muscular disorders, deafness, blindness, periodic paralysis, and channelopathies of the CNS; autism, inflammatory myopathy, and focal segmental glomerulosclerosis (FSGS).

The terms a “disease characterized by autoimmune demyelination” and “demyelinating autoimmune disease” are used interchangeably and refer to a demyelinating disease caused, at least in part, by autoimmune reactions. Demyelinating autoimmune diseases include recurrent or chronically progressive demyelinating diseases, such as multiple sclerosis (MS) and its variants, and monophasic demyelinating diseases, such as optic neuritis, acute disseminated encephalomyelitis, and transverse myelitis. Demyelinating autoimmune diseases of the central nervous system (CNS) include, without limitation, MS and MS variants, such as relapsing remitting MS (RRMS) and primary and secondary progressing forms, and progressive relapsing forms of MS, encephalomyelitis, leukoencephalitis, transverse myelitis, neuromyelitis optica (Devic's disease), and optic neuritis. Demyelinating autoimmune diseases affecting the peripheral nervous system include, for example, acute inflammatory demyelinating polyneuropathy (AIDP; Guillain-Barre syndrome); chronic inflammatory demyelinating polyneuropathy; anti-MAG peripheral neuropathy; and Motor and Sensory Neuropathy (HMSN), also known as Hereditary Sensorimotor Neuropathy (HSMN), or Peroneal Muscular Atrophy, or Charcot-Marie-Tooth Disease.

“Treatment” refers to both therapeutic treatment and prophylactic or preventative measures. Those in need of treatment include those already with the disorder as well as those in which the disorder is to be prevented. Hence, the mammal to be treated herein may have been diagnosed as having the disorder or may be predisposed or susceptible to the disorder. For example, prophylactic treatment includes prevention of a fully developed clinical form, or a more severe form of a disease, such as prevention of the development of MS from relapsing remitting MS (RRMS). Therapeutic treatment may aim at slowing down the progression of the disease, reducing the frequency of episodes of the disease (attacks), return function after an attack, prevent new attacks, and prevent or slowing down the development of disabilities associated with or resulting from the disorder.

The term “CLM-1 agonist” is used herein in the broadest sense, and includes any molecule that partially or fully enhances, stimulates or activates one or more biological activities of CLM-1, in vitro, in situ, or in vivo. For instance, the agonist may function to partially or fully enhance, stimulate or activate one or more biological activities of CLM-1, in vitro, in situ, or in vivo as a result of its direct binding to CLM-, which causes receptor activation or signal transduction. The agonist may also function indirectly to partially or fully enhance, stimulate or activate one or more biological activities of CLM-1, in vitro, in situ, or in vivo as a result of, e.g., stimulating another effector molecule which then causes CLM-1 activation or signal transduction. The biological activity herein is negative regulation of a demyelinating disease, such as a demyelinating autoimmune disease, as hereinabove defined. Agonists specifically include CLM-1 ligands and agonist antibodies to CLM-1.

“Mammal” for purposes of treatment refers to any animal classified as a mammal, including humans, non-human higher primates, domestic and farm animals, and zoo, sports, or pet animals, such as dogs, horses, cats, cows, etc. Preferably, the mammal is human.

The term “therapeutically effective amount” refers to an amount of a drug effective to treat a disease or disorder in a mammal. In the present case, the therapeutically effective amount is an amount of a CLM-1 agonist effective to treat (including prevention) of a demyelinating disease, such as a demyelinating autoimmune disease, as hereinabove defined.

A “liposome” is a small vesicle composed of various types of lipids, phospholipids and/or surfactant which is useful for delivery of a drug (such as the anti-ErbB2 antibodies disclosed herein and, optionally, a chemotherapeutic agent) to a mammal. The components of the liposome are commonly arranged in a bilayer formation, similar to the lipid arrangement of biological membranes.

The term “package insert” is used to refer to instructions customarily included in commercial packages of therapeutic products, that contain information about the indications, usage, dosage, administration, contraindications and/or warnings concerning the use of such therapeutic products.

### II. Detailed Description

Multiple Sclerosis (MS) and its preclinical equivalent Experimental Autoimmune Encephalomyelitis (EAE) are marked by perivascular inflammation and demyelination. Myeloid cells, derived from circulating progenitors, are prominent components of the inflammatory infiltrate and are believed to constitute the ultimate effector cells responsible for cytokine production, demyelination, axonal damage and motor-dysfunction. How the cytotoxic activity of these myeloid cells is regulated is poorly understood. The present invention is based, at least in part, on identifying the Cmrf-Like Molecule-1 (CLM-1) as a negative regulator of autoimmune demyelination. CLM-1 is expressed on inflammatory monocytes in peripheral blood and on inflammatory dendritic cells present in demyelinating areas of the CNS following immunization of mice with MOG peptide. Absence of CLM-1 on CNS infiltrating inflammatory dendritic cells resulted in significantly increased nitric oxide and proinflammatory cytokine production, along with increased axonal demyelination and worsened clinical scores, while T-cell responses remain unaffected. Therefore CLM-1 is identified herein as a negative regulator of myeloid cell activation and autoimmune demyelination.

Myeloid cells are the primary effector cells in autoimmune demyelinating diseases (Barnett et al., Multiple sclerosis (Houndmills, Basingstoke, England) 12, 121-132, 2006; Benveniste, Journal of Molecular Medicine (Berlin, Germany) 75, 165-173, 1997). The CNS-infiltrating myeloid population consists of resident microglia, macrophages, inflammatory dendritic cells, plasmacytoid dendritic cells and conventional dendritic cells. MHCII and CD86 expressing myeloid dendritic cells (DCs) have received special attention due to their ability to reactivate antigen-specific T-cells (Deshpande et al., J Immunol 178, 6695-6699, 2007) and their involvement in epitope spreading leading to relapsing disease (Miller et al., Annals of the New York Academy of Sciences 1103, 179-191, 2007). Next to serving as antigen presenting cells, inflammatory DCs directly regulate the local extracellular milieu by secreting proinflammatory cytokines and reactive oxygen intermediates, resulting in progressive demyelination and axon loss. The precursor cells of these TNF- and iNOS producing dendritic cells, also named TipDCs (Serbina et al., Immunity 19, 59-70, 2003) are inflammatory monocytes present in the circulation and recruited to areas of CNS inflammation. Converting inflammatory to type II anti-inflammatory monocytes by glatiramer acetate, a drug approved for MS, resulted in reversion of EAE severity (Weber et al., Nature Medicine 13, 935-943, 2007), further stressing an important role of these myeloid cells in regulating disease severity.

Other negative regulators of CNS infiltrating myeloid cells have previously been identified. For example, TREM-2 expressed on both resident microglia and infiltrating myeloid cells plays an important role in resolution of CNS inflammation by phagocytosis of myelin debris (Piccio et al., European Journal of Immunology 37, 1290-1301, 2007) (Takahashi et al., PLoS medicine 4, e124, 2007) (Takahashi et al., The Journal of Experimental Medicine 201, 647-657, 2005). Similarly, IFNAR on myeloid cells down-modulates inflammatory responses in the CNS (Prinz et al., Immunity 28, 675-686, 2008). However, neither receptor is specific for inflammatory bone marrow-derived monocytes homing to the CNS.

CLM-1 (MAIR-V, LMIR-3, DigR2) was identified in search for myeloid specific cell surface receptors important for negative regulation of myeloid function. CLM-1 is part of the CMRF family, a multigene cluster on human chtromosome 17 with the mouse orthologues located on chromosome 11. All family members contain an extracellular IgV domain. Two family members in this cluster (CLM-1 and CLM-8) contain an ITIM sequence in the intracellular domain, the remainder have charged residues in the transmembrane region that may serve to recruit signaling adapters. CLM-1 (SEQ ID NO: 1), the murine orthologue of human CD300f (SEQ ID NO: 2; Clark et al., Trends in Immunology 30, 209-217, 2009), was first described as a negative regulator of osteoclastogenesis (Chung et al., J Immunol 171, 6541-6548, 2003). Subsequent studies have shown that CLM-1 serves an inhibitory role in Fc-receptor-mediated cell responses (Alvarez-Errico et al., 2004; Fujimoto et al., 2006). A biological role in autoimmune disease so far has not been described. Here we identify CLM-1, as a negative regulator of inflammatory DCs activity in the CNS by suppressing release of inflammatory cytokines and reactive oxygen species. This study thus identifies CLM-1 as a myeloid specific negative regulator of CNS inflammation and demyelination.

The present invention concerns methods for the diagnosis and treatment of demyelinating diseases, such as demyelinating autoimmune diseases, with CLM-1 antagonists.

In a specific embodiment, the CLM-1 agonist is an agonist antibody to CLM-1.

### Antibodies

Antibodies of the invention include anti-CLM-1 antibodies or antigen-binding fragments of CLM-1, or other antibodies described herein. Exemplary antibodies include, e.g., polyclonal, monoclonal, humanized, fragment, multispecific, heteroconjugated, multivalent, effector function, etc., antibodies. In certain embodiments of the invention, the antibody is an agonist antibody.

### Polyclonal Antibodies

The antibodies of the invention can comprise polyclonal antibodies. Methods of preparing polyclonal antibodies are known to the skilled artisan. For example, polyclonal antibodies against CLM-1 are raised in animals by one or multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, or SOCl₂.

Animals are immunized against CLM-1, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Typically, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

### Monoclonal Antibodies

Monoclonal antibodies can be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that typically contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Typical myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against CLM-1. The binding specificity of monoclonal antibodies produced by hybridoma cells can be determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

In another embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

### Humanized and Human Antibodies

Antibodies of the invention can comprise humanized antibodies or human antibodies. A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a typical method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992). Human antibodies can also be derived from phage-display libraries (Hoogenboom et al., J. Mol. Biol., 227:381 (1991); Marks et al., J. Mol. Biol., 222:581-597 (1991); Vaughan et al. Nature Biotech 14:309 (1996)).

Human antibodies can also be produced using various techniques known in the art, including phage display libraries (Hoogenboom and Winter, J. Mol. Biol., 227:381 (1991); Marks et al., J. Mol. Biol., 222:581 (1991)). According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, K S, and Chiswell, D J., Cur Opin in Struct Biol 3:564-571 (1993). Several sources of V-gene segments can be used for phage display. For example, Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated, e.g., by essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905. The techniques of Cole et al. and Boerner et al. are also available for the preparation of human monoclonal antibodies (Cole et al., Monoclonal Antibodies and Cancer Therapy, Alan R. Liss, p. 77 (1985) and Boerer et al., J. Immunol., 147(1):86-95 (1991)). Human antibodies may also be generated by in vitro activated B cells (see U.S. Pat. Nos. 5,567,610 and 5,229,275).

### Antibody Fragments

Antibody fragments are also included in the invention. Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′).sub.2 fragments (Carter et al., Bio/Technology 10: 163-167 (1992)). According to another approach, F(ab′).sub.2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. SFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

### Multispecific (e.g., Bispecific) Antibodies

Antibodies of the invention also include, e.g., multispecific antibodies, which have binding specificities for at least two different antigens. While such molecules normally will only bind two antigens (i.e. bispecific antibodies, BsAbs), antibodies with additional specificities such as trispecific antibodies are encompassed by this expression when used herein.

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in WO96/27011, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab'-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol., 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V.sub.H) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

### Heteroconjugate Antibodies

Bispecific antibodies include cross-linked or “heteroconjugate” antibodies, which are antibodies of the invention. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

### Multivalent Antibodies

Antibodies of the invention include a multivalent antibody. A multivalent antibody may be internalized (and/or catabolized) faster than a bivalent antibody by a cell expressing an antigen to which the antibodies bind. The antibodies of the invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

### Effector Function Engineering

It may be desirable to modify the antibody of the invention with respect to effector function, so as to enhance the effectiveness of the antibody in treating disease, for example. For example, a cysteine residue(s) may be introduced in the Fc region, thereby allowing interchain disulfide bond formation in this region. The homodimeric antibody thus generated may have improved internalization capability. See Caron et al., J. Exp Med. 176:1191-1195 (1992) and Shopes, B. J. Immunol. 148:2918-2922 (1992). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG.sub.1, IgG.sub.2, IgG.sub.3, or IgG.sub.4) that is responsible for increasing the in vivo serum half-life of the IgG molecule.

### Other Antibody Modifications

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

### Liposomes and Nanoparticles

The CLM-1 antibodies of the invention may also be formulated as immunoliposomes. Liposomes containing the polypeptide are prepared by methods known in the art, such as described in Epstein et al., Proc. Natl. Acad. Sci. USA, 82:3688 (1985); Hwang et al., Proc. Natl. Acad. Sci. USA, 77:4030 (1980); and U.S. Pat. Nos. 4,485,045 and 4,544,545. Liposomes with enhanced circulation time are disclosed in U.S. Pat. No. 5,013,556. Generally, the formulation and use of liposomes is known to those of skill in the art.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A polypeptide of the invention can be conjugated to the liposomes as described in Martin et al. J. Biol. Chem. 257: 286-288 (1982) (e.g., Fab′ fragments of an antibody) via a disulfide interchange reaction. Nanoparticles or nanocapsules can also be used to entrap the polypeptides of the invention. In one embodiment, a biodegradable polyalky-cyanoacrylate nanoparticles can be used with the polypeptides of the invention.

Further details of the invention are illustrated by the following non-limiting Examples. The disclosures of all citations in the specification are expressly incorporated herein by reference.

### EXAMPLE

### Materials and Methods

Animals. All animals were held under Sterile Pathogen Free conditions and animal experiments were approved by the Institutional Animal Care and Use Committee of Genentech. To generate Clm-1 knock-out (KO) mice, a linearized targeting vector containing a neomycin-resistance gene (Neo^{r}) was electroporated into C2 embryonic stem (ES) cells of C57B1/6 origin. Neomycin resistant ES clones were selected for Southern blotting analysis of homologous recombination (Supplemental Fig). ES clones with successful replacement of Clm-1 exon 1 with the Neo^{r} gene were injected into C57BL/6 blastocytes and subsequently transferred into pseudopregnant females to generate chimeric offspring. Chimeras were bred with C57BL/6 mice to produce heterozygotes. Heterozygotes with germline transmission of the targeted allele were backcross to C57BL/6 for at least 10 generations before interbred to generate Clm-1 wild-type (WT) and KO mice. C57BL/6 (on CD45. 1 or CD45.2 congenic backgrounds) mice were purchased from The Jackson Laboratory. Cx3cr1^{gfp/+} C57BL/6 reporter mice were bred and maintained in pathogen-free animal facility of Genentech, Inc. All mice were used at the age of 8-12 wk old except for the CD45.1/CD45.2 bone-marrow chimeria experiment where 6-wk-old C57BL/6 (CD45.1) were used as bone-marrow recipients. All experimental protocols were approved by the Institutional Animal Care and Use Committee of Genentech, Inc.

Antibodies and recombinant proteins. The following antibodies were purchased from BD Biosciences: anti-FcγRIII/II (CD32/16, clone 2.4G2); PE-, APC-, APC-Cy7-labeled anti-CD11b (M1/70); Biotin-, PE-, APC-labeled anti-CD11c (HL3); PE-, APC-labeled anti-CD4 (GK1.5); APC-labeled anti-CD3 (145-2C11); PE-Cy7-labeled anti-B220 (RA3-6B2); PE-labeled anti-I-A/I-E (M5/114.15.2); Biotin-, PE-labeled anti-CD86 (GL1); APC-Cy7-labeled anti-Gr-1 (RB6-8C5); PE-labeled anti-CD45.1 (A20); Biotin-, FITC-labeled anti-CD45.2 (104); Alexa Fluor 488-labeled anti-FoxP3 (MF23); PE-labeled anti-IL-17 (TC11-18H10); FITC-labeled anti-IFN□ (XMG1.2); FITC-, PE-labeled anti-TNFα (MP6-XT22); Biotin-, PE-, PerCP-Cy5.5-labeled anti-CD45 (30-F11); Polyclonal rabbit anti-iNOS type II antibody. The following antibodies were purchased from eBioscience: Pacific blue-labeled anti-CD11b (M1/70); PE-Cy7-labeled anti-CD11c (N418); PE-Cy5-labeled anti-I-A/I-E (M5/114.15.2); APC-Alexa Fluor 750-labeled anti-F4/80 (BM8). Streptavidin Pacific Orange was purchased from Invitrogen. PE-labeled donkey anti-rabbit IgG and Cy3-labeled anti-hamster IgG were purchased from Jackson ImmunoResearch. Monoclonal anti-Actin antibody (AC-40) was purchased from Sigma-Aldrich. To generate murine Clm-1-Fc fusion protein, the extracellular domain (ECD) of murine Clm-1 was cloned into a modified pRK5 expression vector encoding the murine IgG1 Fc fragment. The expression vector was transfected into CHO cells and the Clm-1-Fc fusion protein contained in the cell culture supernatants was purified by protein A affinity chromatography and subsequent Superdex 200 gel filtration. The identity of the purified protein was verified by mass spectrometry analysis and the endotoxin level was <0.05 EU/mg. The murine anti-gp120 antibody (IgG1) was used as a control. Monoclonal antibodies to ECD of murine Clm-1 were generated by immunizing Armenian hamsters with murine Clm-1-ECD-His fusion proteins. Splenic B cells from immunized animals were fused to myelomas to generate hybridomas. Positive clones were selected based on the reactivity to murine Clm-1 by ELISA, FACS, Western blotting and immunohistochemistry analyses. The clone 3F6 was selected for use in the study based on the above criteria. Alexa fluorochrome (488 or 647)-conjugated Clm-1 antibodies were generated using the Alexa Fluor® protein labeling kits (Invitrogen).

Active induction of EAE and clinical evaluation Mice were immunized subcutaneously with 200 μg of MOG₃₅₋₅₅ peptide in 200 μl of an emulsion containing 100 μl of PBS and 100 μl of complete Freund's adjuvant (CFA). CFA is prepared by mixing incomplete Freund's adjuvant (DIFCO Laboratories) with 8 mg/ml of Mycobacterium tuberculosis H37RA (nonviable and desiccated; DIFCO Laboratories). Each mouse was also injected intraperitoneally with 200 ng pertussis toxin (Calbiochem) in 100 μl of PBS on days 0 and 2 post-immunization. Clinical signs were evaluated using the following grading system: 0, no abnormality; 1, limp tail or hind limb weakness; 2, limp tail and hind limb weakness; 3, partial hind limb paralysis; 4, complete hind limb paralysis; 5, moribund state. For Clm-1-Fc fusion protein experiment, starting on day 0 of immunozation, mice were treated subcutaneously three times weekly with 200 μg of Clm-1-Fc fusion protein in 100 μl PBS or a control Fc protein (anti-gp120). Data are reported as the mean daily clinical score and standard error of the mean (SEM).

Bone marrow chimeras Six-week-old C57BL/6 (CD45.1) recipient mice were lethally irradiated with two doses of 500 rad each. Bone marrow cells from femur and tibia were harvested aseptically from C57BL/6 (CD45.2) donor mice by flushing the bones with Hanks balanced salt solution (HBSS; Hyclone) containing 5% FBS with a syringe and a 27-gauge needle. Erythrocytes were lyzed by ACK lysis buffer. The cells were washed in HBSS/FBS at 400 g for 5 minutes, resuspended, and passed through a nylon mesh (BD Falcon) to remove debris. The cells were then washed twice with PBS and resuspended at a concentration of 10⁸ cells/ml. Irradiated recipient mice were injected with 2×10⁷ cells/200 μl via tail vein. The reconstituted mice were maintained in a pathogen-free facility for 8 weeks to allow for complete engraftment with donor bone marrow. Full reconstitution of bone marrow was verified by FACS analysis of peripheral blood for CD45.1 and CD45.2 congenic markers in lymphoid and myeloid compartments. EAE was induced in the reconstituted recipient mice as described above.

Adoptive transfer of EAE Clm-1 WT or KO mice were immunized with MOG₃₅₋₅₅ peptide as described for the induction of active EAE except that mice were not injected with pertussis toxin. Ten to twelve days postimmunization, draining (inguinal and brachial) lymph nodes were harvested and single-cell suspensions were obtained by mashing through 70-μm cell strainers. Cells were restimulated for 4 days with 20 μg/ml of MOG₃₅₋₅₅ peptide and 20 ng/ml of recombinant murine IL-2 (R&D Systems) at 5×10⁶ cells/ml in complete medium (RPMI 1640, 10% FBS, 2 mM glutamine, 10 mM HEPES, 1 mM sodium pyruvate, 0.05 mM β-mercaptoethanol, 100 U/ml penicillin, 100 mg/ml streptomycin). Recipient mice were injected with 10⁷ cells via tail vein. At the same day and two days later, the recipient mice will also be injected with 200 ng pertussis toxin as described above. Clinical evaluation of EAE disease was performed as described above.

FACS analysis of spinal cord and lymph node At the peak of EAE disease (day 14-15 post-immunization), mice were anaesthetized and perfused transcardially with PBS containing 10 U/ml heparin. Spinal cords were dissected and digested with collagenase D (2 mg/ml; Roche Diagnostics). Mononuclear cells were isolated by passing the tissue through 70-μm cell strainers (BD Biosciences) followed by Percoll gradient (80%/70%/60%/30%) centrifugation. Cells were collected from the 30%/60% interface and washed. Cells were also isolated from draining lymph nodes (DLNs) as described above. Cells were Fc blocked with anti-Fc□RIII/II at 4° C. for 30 min in FACS staining buffer (PBS, 0.5% bovine serum albumin, 2 mM EDTA). After washing, cells were stained with fluorescent conjugated mAbs at 4° C. for 30 min. For intracellular staining of iNOS, cells were stained with antibodies to Clm-1 (3F6), CD45, CD11b and CD11c followed by fixation with 3% Paraformaldehyde in PBS solution at room temperature for 20 min. Cells were then resuspended in 100 μl permeabilization solution (0.1% Triton-X in PBS). Cells were stained with 1 μg/ml rabbit anti-iNOS antibody in permeablization solution at room temperature for 15 min followed by staining with PE-labeled donkey anti-rabbit IgG at room temperature for 15 min. To analyze Treg cells, single cells suspensions isolated from spinal cords and DLNs as described above were stained with CD45 and CD4 followed by intracellular staining of FoxP3 using the Cytofix/Cytoperm Fixation/Permeabilization kit (BD Biosciences) according to the manufacturer's instructions. For intracellular staining of cytokines, cells were stimulated at 37° C. for 18-20 hrs with 100 μg/ml MOG₃₅₋₅₅ peptide at 4×10⁵ cells/200 μl complete medium in a 96-well round-bottom plate. During the final 4 hrs of stimulation, cells were treated with GolgiPlug (BD Biosciences) at dilution of 1:1000. Intracellular staining of IL-17 and IFN□ was performed essentially as FoxP3 staining. The stained cells were analyzed using a FACSCaliber or LSRII flow cytometer (Becton Dickinson). Data were analyzed using the FlowJo software (Tree Star).

Clm-1 expression by Western blot Bone marrow-derived dendritic cells (BMDCs) were generated as described (Inaba et al., The Journal of Experimental Medicine 176, 1693-1702, 1992). BMDCs were cultured in the presence of 10 ng/ml GM-CSF (R&D Systems) with medium refreshment every three days. On day 7, cells were analyzed by FACS. The BMDC purity was 90-95% CD11c⁺, CD11b⁺. Total cell lysates from BMDCs were analyzed by immunoblotting with anti-Clm-1 Ab (3F6) using standard methods.

Real-time PCR analysis of Clm-1 expression Spinal cords and DLNs were isolated from mice on Day 0, 7, 14 and 21 of EAE as described above. Total RNA was isolated using the RNeasy Protect Mini Kit (QIAGEN). cDNA was synthesized with 1 μg RNA using the High-Capacity cDNA Reverse Transcription kit (Applied Biosystems). Clm-1 mRNA and 18s rRNA were measured using the TaqMan Universal PCR Master Mix and verified primer and probe sets, Mm00467508_ml and Hs03003631_gl, respectively (Applied Biosystems).

Measurement of cytokine and nitric oxide production Mononuclear cells were isolated from spinal cords on Day 15 of EAE as described above. Single-cell suspensions were cultured at 37° C. in complete medium (5×10⁵ cells/200 μl) with or without 100 μg/ml MOG₃₅₋₅₅ peptide in a 96-well round-bottom plate. Culture supernatants were harvested after 36 hrs. Cytokine release was measured by Luminex using the Bio-Plex mouse cytokine 23-plex panel (Bio-Rad). Nitric oxide production was measured using the Griess assay (Promega) according to the manufacturer's instructions.

In vitro antigen-specific recall responses Draining lymph nodes were harvested from mice on Day 14 of EAE as described above. Single-cell suspensions were re-stimulated at 37° C. for 3 days in complete medium (5×10⁵ cells/200 μl) with or without titrated amount of MOG₃₅₋₅₅ peptide in a 96-well round-bottom plate. Cells were then pulsed with 0.5 μCi/well [³H] Thymidine for the final 6 hrs of culture. Proliferation was determined by uptake of [³H] Thymidine detected using a Topcount Microplate Scintillation Counter (Packard Instruments). Alternatively, supernatants were collected at 3 days for cytokine analysis. Cytokine measurements were performed by ELISA (BD Biosciences).

Immunohistochemistry On the indicated days post-immunization, mice were anesthetized and perfused with 30 ml PBS as described above followed by perfusion with 10 ml 4% paraformaldehyde (PFA). Spinal cords were removed by dissection and fixed overnight in 4% PFA followed by submersion in 10%, 20%, 40% sucrose solution subsequently. The spinal cords were then frozen in OCT on dry ice and stored at −80° C. in plastic bags to prevent dehydration. Seven-micrometer thick cross-sections were cut and mounted on Superfrost Plus slides (Fisher Scientific). For Clm-1 and CD45.2 co-staining, slides were blocked using hamster serum and biotin blocking kit (Sigma). Tissues were stained with hamster anti-Clm-1 (3F6) and biotin-conjugated anti-CD45.2 followed by detection with Cy3-anti-hamster IgG and Alexa Fluor 488-streptavidin (Invitrogen). For Clm-1 and CD11c co-staining, slides were first stained with anti-Clm-1 followed by detection with Cy3-anti-hamster IgG. The slides were then stained with Biotin-anti-CD11c (HL3) and detected with Alexa Fluor 488-Streptavidin. For myelin and CD11c co-staining, slides were first stained with Biotin-anti-CD11c and detected with Alexa Fluor 594-Streptavidin (Invitrogen). Myelin was then stained using the FluoroMyelin™ Green Fluorescent Myelin Stain kit (Invitrogen). Sections were coverslipped with Prolong Gold antifade medium with DAPI (Invitrogen). Slides were examined and images were acquired using the Olympus BX61 fluorescent microscope. To determine degree of demyelination, cervical and thoracic sections of spinal cords were stained with FluoroMyelin™ Green Fluorescent Myelin Stain kit. Areas of demyelination were assessed by manually tracing the total cross-sectional area and the demyelinated area of each section. Total demyelination was expressed as a percentage of the total spinal cord area.

Statistical Analyses Comparison of EAE clinical scores, demyelination or other cell counts and cytokine production between any two groups of mice was done by two-tailed paired student's t test, assuming unequal variance. p values <0.05 were considered significant.

### Results and Discussion

Clm-1 is expressed on TNF and iNOD producing CD11c+ cells at sites of CNS inflammation

Clm-1 was first identified through a bio-informatics approach searching genomic predicted sequences coding for single trans-membrane, Immune-Tyrosine Inhibition Motif (ITIM)-containg Ig-superfamily members (Abbas et al., Genes and Immunity 6, 319-331, 2005). Mouse homologues of the candidate ITIM-containing genes where then selected based on changes in expression levels in the spinal chord following immunization with a Myelin Oligodendrocyte Glycoprotein (MOG) peptide. Expression of Clm-1 was increased over 100 fold at peak disease compared to naïve mice (FIG. 1A, left panel). Monoclonal antibodies to CLM-1 extracellular domain were generated to determine the cellular source of CLM-1. CLM-1 was absent on the local microglia population in naive mice (FIG. 1B). In spinal chords from MOG-immunized mice, CLM-1 was expressed on CD11b/CD11c double positive cells with high MHC class II and CD86 expression (FIG. 1C). At disease onset, CLM-1 CD11c double positive cells were distributed along meninges and blood vessels (results not shown). At peak of disease, Clm-1+ cells were located in clusters in white matter of the dorsal and ventral horn of the thoracic and lumbar spinal chord (FIG. 1D). Further analysis showed that Clm-1+ cells expressed iNOS and TNF (FIG. 1E) and therefore phenotypically resemble Tip-DCs, first described as a subset of myeloid cells required for efficient pathogen elimination (Serbina et al., Immunity 19, 59-70, 2003). Subsequent studies in EAE has identified the TipDCs and their precursors as pathogenic effector cells contributing to disease pathogenesis in EAE (King et al., Blood 113, 3190-3197, 2009). The increased expression of CLM-1 on inflammatory myeloid cells in the CNS may therefore indicate a modulatory function in EAE disease pathogenesis.

CLM-1 is expressed on circulating Ly6+ myeloid precursors migrating to the CNS during autoimmune demyelinating disease

To further determine the myeloid lineage from which the CLM-1 positive cells are derived, we made use of a Cx3cr1+/gfp reporter strain which expresses green fluorescent protein in cells of the monocyte and macrophage/dendritic cells lineage (Geissmann et al., Immunity 19, 71-82, 2003). In peripheral blood, CLM-1 was expressed on Cx3cr1^{lo} Ly6C^{hi} CD115⁺CD62L⁺Ly6G⁻ inflammatory monocytes following MOG immunization but was absent on Cx3cr1^{hi}CD11c⁺ common DC precursors in naïve and immunized mice (FIG. 2A) (Auffray et al., The Journal of Experimental Medicine 206, 595-606, 2009; Liu et al., Science, 324, 392-397, 2009). To further determine if the CLM-1 positive cells in the inflamed CNS indeed originate from irradiation-sensitive bone-marrow derived cells and not from irradiation-resistant CNS microglia, mice with the CD45.1 allotype were irradiated and reconstituted with donor cells with the CD45.2 allotype. CLM-1 expression was absent on irradiation-resistant microglia, but present on bone marrow-derived donor cells homing to the CNS (FIG. 2B). Confirming these results, CLM-1 was absent on Cxcr3^{hi} resident microglia cells of the naïve spinal chord, but highly expressed on a subpopulation of Cx3cr1⁺CD11c⁺ cells at peak of disease (FIG. 2C). CLM-1⁺Cx3cr1^{lo} double positive cells were found adjacent to meninges of the dorsal and ventral horn of the thoracic and lumbar spinal chord as well as the median eminence, but remained absent on resident microglia cells located in the grey matter of the dorsal and ventral horn of the spinal chord (FIG. 2D). Taken together, these results indicate that CLM-1 is expressed on inflammatory monocytes and bone marrow-derived inflammatory DCs in CNS inflammatory lesions, but not on circulating common DC precursors or CNS resident microglia.

Absence of CLM-1 results in increased disease severity in MOG-induced EAE

As CLM-1 contains two ITIM and one ITSM motif in its cytoplasmic domain (Chung et al., J Immunol 171, 6541-6548, 2003) and is able to recruit SHP-1 following cross-linking with an activating receptor in forced overexpression systems (Izawa et al., The Journal of Biological Chemistry 282, 17997-18008, 2007), we determined if CLM-1 could serve to inhibit inflammatory responses in MOG-induced EAE. Mice were generated that lacked exon 1 of CLM-1 through homologous recombination, resulting in the absence of transcript and protein (Supplemental FIG. 1). CLM-1 ko mice were viable and born in the expected Mendelian ratios. Mice did not differ in weight or bone parameters measured at 6, 9 and 12 weeks of age (results not shown). Myeloid and lymphoid cell subsets in the inguinual lymph nodes, spleens and blood were similar in CLM-1 ko and wt mice (results not shown). Successful ablation of the CLM-1 gene in the ko mice was confirmed by flow cytometry and Western blot analysis (FIG. 3A, left panels). Expression levels of cell surface molecules associated with antigen presentation and co-stimulation were similar on bone marrow-derived DCs (BMDCs) from CLM-1 wt and ko mice (FIG. 3A, right panels) as was the expression level of other members in the CMRF cluster (results not shown). Dendritic cell morphology (FIG. 3B, left panel) and numbers of various inflammatory cell populations at peak of disease (FIG. 3B, right panel) were similar in CLM-1 wt and ko mice. Upon MOG immunization, both CLM-1 wt and ko mice developed disease with similar incidence. However, disease severity was significantly increased in mice lacking CLM-1 (FIG. 3C). To determine if this phenotype was due to the engagement of CLM-1 by a putative ligand, mice were treated with a soluble version of CLM-1 (CLM-1-Fc fusion protein). Consistent with the results obtained in CLM-1 ko mice, disease severity was significantly increased in CLM-1-Fc treated mice as compared to mice treated with a control fusion protein, while disease incidence remained similar (FIG. 3D). Thus, lack of CLM-1 receptor function leads to increased disease severity pointing to a potential inhibitory role in CNS inflammation.

Clm-1 does not regulate T-cell priming

A spliced variant of CLM-1, Digr1, was previously identified as a negative regulator of

T-cell responses (Shi et al., Blood 108, 2678-2686, 2006). Since EAE can be induced by antigen-specific T-cell priming, we further determined if CLM-1 influences T-cell responses. Splenic cDCs or BMDCs derived from CLM-1 wt and ko mice were incubated with allogeneic T-cells or with T cells expressing a TCR specific for OVA peptide. Proliferation (Supplemental FIG. 2) and cytokine responses (results not shown) did not depend on CLM-1 status. To further determine if CLM-1 influences T-cell priming in vivo, T-cells isolated from peripheral lymph nodes 7 days following MOG immunization were isolated and re-stimulated with MOG peptide. CLM-1 status did not influence T-cell proliferation, cytokine responses or generation of Foxp3 regulatory T-cells in peripheral lymph node (PLN) cells (FIG. 4A and Supplemental FIG. 3a). Finally, to consolidate a role for CLM-1 in regulating T-cell effector functions in vivo, T cells from CLM-1 wt and ko donors were adoptively transferred into ko and wt recipients, respectively. Disease severity was not influenced by CLM-1 status in the T-cell donor, but was significantly enhanced in T-cell recipients lacking CLM-1 (FIG. 4B). This indicates that CLM-1 acts to regulate disease severity at the effector phase, and not the initial T-cell priming phase, following MOG immunization.

Next, we determined whether CLM-1 influenced re-activation of CNS infiltrating CD4+ T-cells and cytotoxic activity of inflammatory DCs. CNS leukocytes harvested from the spinal chord at peak of disease and re-stimulated with MOG peptide in the presence of antigen presenting cells showed similar polarization towards Th1, Th17 and Foxp3 Treg cells and similar T-cell specific cytokine responses (FIG. 5A and Supplemental FIG. 3b). In contrast, leukocytes obtained from spinal chords of CLM-1 ko mice produced significantly elevated levels of nitric oxide and myeloid-specific pro-inflammatory cytokines as compared to wt mice (FIG. 5B). Thus, CLM-1 negatively regulates myeloid effector function in MOG-induced EAE without affecting T-cell responses.

We next determined if increased paralysis of CLM-1 ko mice immunized with MOG peptide resulted from enhanced activity of myeloid cells in the spinal chord. CLM-1 positive cells were found clustered at sites of demyelination in the dorsal horn of the cervical and thoracic spinal chord. The cells were found apposed to-, and often wrapped around-, myelin sheets (FIG. 6A) with in some cases MOG-positive myelin remnants present inside CLM-1 positive cells (results not shown). Since CLM-1 is an inhibitory receptor and the degree of infiltrating myeloid cells is similar in CLM-1 wt and ko mice, we reasoned that absence of CLM-1 could result in increased activation and effector activity per cell, resulting in increased demyelination. Lack of CLM-1 resulted in increased demyelination (FIGS. 6B and C) indicating increased cytotoxic activity in CD11c+ cells lacking CLM-1. Thus, CLM-1 negatively regulates myeloid cell activation, putting the breaks on axonal demyelination in the spinal chord.

While the number of receptors containing an ITIM-sequences in their intracellular domain is steadily increasing, the biological role for many of these receptors is still poorly understood. This study for the first time identifies CLM-1 as an inhibitory receptor on CNS infiltrating inflammatory DCs. We further show that a soluble version of the receptor can exacerbate disease severity, suggesting that the extracellular domain serves as a decoy receptor for an as yet to be identified ligand. While identification of a putative ligand will undoubtly increase our understanding of CLM-1 biology, this study clearly illustrates that CLM-1 plays a non-redundant role in controlling myeloid cell activation and demyelination in the CNS.

1. A method for the treatment of a demyelinating disease in a mammalian subject comprising administering to said subject an effective amount of a CLM-1 agonist.

2. The method of claim 1 wherein the mammalian subject is a human

3. The method of claim 2 wherein the demyelinating disease is a demyelinating autoimmune disease.

4. The method of claim 3 wherein the demyelinating autoimmune disease affects the central nervous system (CNS).

5. The method of claim 4 wherein the demyelinating autoimmune disease is selected from the group consisting of multiple sclerosis (MS), relapsing remitting MS (RRMS), primary and secondary progressing forms of MS, progressice relapsing forms of MS, encephalomyelitis, leukoencephalitis, transverse myelitis, neuromyelitis optica (Devic's disease), and optic neuritis.

6. The method of claim 5 wherein the demyelinating autoimmune disease is MS.

7. The method of claim 3 wherein the demyelinating autoimmune disease affects the periopheral nervous system.

8. The method of 7 wherein the demyelinating autoimmune disease is selected from the group consisting of acute inflammatory demyelinating polyneuropathy (AIDP; Guillain-Barre syndrome); chronic inflammatory demyelinating polyneuropathy; anti-MAG peripheral neuropathy; and Motor and Sensory Neuropathy (HMSN) (also known as Hereditary Sensorimotor Neuropathy (HSMN), or Peroneal Muscular Atrophy, or Charcot-Marie-Tooth Disease).

9. The method of any one of claims 1 to 8 wherein the CLM-I agonist is an agonist anti-CLM-1 antibody.

10. A pharmaceutical composition for the treatment of a demyelinating disease, comprising an effective amount of a CLM-I agonist in admixture with a pharmaceutically acceptable excipient.

11. The pharmaceutical composition of claim 10 wherein the demyelinating disease is a demyelinating autoimmune disease.

12. The pharmaceutical composition of claim 10 wherein the demyelinating autoimmune disease is multiple sclerosis (MS).

13-20. (canceled)

21. A method for the diagnosis of a demyelinating disease comprising detecting a defect in the function of CLM-I.

22. The method of claim 21 wherein the demyelinating disease is a demyelinating autoimmune disease.

23. The method of claim 22 wherein the demyelinating autoimmune disease is MS.

24. A kit comprising a CLM-I agonist and instructions for the treatment of a demyelinating disease.

25. The kit of claim 24 wherein the demyelinating disease is a demyelinating autoimmune disease.

26. The kit of claim 25 wherein the demyelinating autoimmune disease is MS.
